(19) United States
(12) Patent Application Publication
(10) Pub. No.: US 2022/0162152 A1
(43) Pub. Date: May 26, 2022

(54) Preparation of polyalkylene-polyamines by reductive amination

(71) Applicant: BASF SE, Ludwigshafen am Rhein (DE)

(72) Inventors: Matthaeus Kopczynski, Ludwigshafen (DE); Dieter Kolassa, Ludwigshafen (DE); Hannes Ferdinand Zipfel, Ludwigshafen (DE); Ansgar Gereon Altenhoff, Ludwigshafen (DE); Christian Eidamshaus, Ludwigshafen (DE)

(73) Assignee: BASF SE, Ludwigshafen am Rhein (DE)

(21) Appl. No.: 17/434,815

(22) PCT Filed: Mar. 2, 2020
(86) PCT No.: PCT/EP2020/055393

§ 371 Date: Aug. 30, 2021

(30) Foreign Application Priority Data
Mar. 5, 2019 (EP) 19160801.7

(51) Int. Cl. C07C 209/26; B01J 23/72

(52) U.S. Cl. CPC C07C 209/26; B01J 23/72

(57) ABSTRACT

An alkylene-diamine, a hydroxyalkylene-amine, and hydrogen are reacted in presence of a heterogenous catalyst containing copper to produce polyalkylene-polyamines.

Object of the present invention is a process to produce polyalkylene-polyamines wherein an alkylene-diamine, a hydroxyalkylene-amine and hydrogen are reacted in presence of an heterogenous catalyst comprising copper.

Compounds with primary or secondary amino groups are valuable intermediates in chemical synthesis. Furthermore, they are important monomers and catalysts in the technical field of polymers.

Polyalkylene-polyamines are of specific interest due to their high content of primary and secondary amino groups.

According to K. V. Chernitskii and V. A. Bobylyev, Zhurnal Obshchei Khimii, 1990, vol. 60 (7), 1636-1642 polyalkylene-polyamines and specifically dimethyl-diethylene-triamine are obtained by amination of 1,2-dihaloalkanes.

U.S. Pat. No. 4,806,517 discloses the synthesis of polyethylene-polyamines by reacting ethylene-diamine and mono-ethanol-amine in presence of a phosphorous catalyst.

The use of copper containing catalysts in reductive amination is known, for example, from EP-B 2802553.

There is an ongoing demand in compounds with high amino functionality that may be prepared by an easy and economic process in high yield and high selectivity.

Accordingly, the above process for the preparation of polyalkylene-polyamines has been found.

To the starting materials

In the process, an alkylene-diamine, a hydroxyalkylene-amine and hydrogen are reacted.

The alkylene-diamine is a compound with an alkylene group and two primary amino groups as substituents to the alkylene group.

The alkylene-diamine is preferably a low molecular weight compound with a molecular weight of at maximum 500 g/mol.

Preferably, the alkylene-diamine does not comprise other atoms than hydrogen, carbon and nitrogen and does not comprise other amino groups than the two primary amino groups.

In a preferred embodiment, the alkylene-diamine is a compound of formula I

H₂N—X—NH₂

with X representing a linear or branched alkylene group with 2 to 10 carbon atoms, notably 2 to 4 carbon atoms.

A specifically preferred alkylene-diamine of formula I is 1,2 propylenediamine of formula

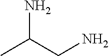

The hydroxyalkylene-amine is a compound with an alkylene group and one hydroxy group and one primary amino group as substituents to the alkylene group.

The hydroxyalkylene-amine is preferably a low molecular weight compound with a molecular weight of at maximum 500 g/mol.

Preferably, the hydroxyalkylene-amine does not comprise any other atoms than hydrogen, carbon, nitrogen and oxygen and does not comprise any other amino groups or any other oxygen containing functional groups.

In a preferred embodiment, the hydroxyalkylene-amine is a compound of formula II

HO—Y—NH₂

with Y representing a linear or branched alkylene group with 2 to 10 carbon atoms, notably 2 to 4 carbon atoms.

Specifically, preferred hydroxyalkylene-amines of formula II are 1-amino propan-2-ol of formula

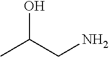

or 2-aminopropan-1-ol of formula

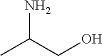

or mixtures thereof.

The starting materials may be prepared in situ. For example, an alkylene-diamine may be obtained by reacting hydroxyalkylene-amine with ammonia. With an excess of hydroxyalkylene-amine or short reaction times mixtures of the two starting materials alkylene-diamine and hydroxyalkylene-amine may be obtained.

To the products obtained

The reaction of the alkylene-diamine, a hydroxyalkylene-amine and hydrogen is a reductive amination resulting in a polyalkylene-polyamine or a mixture of polyalkylene-polyamines.

The expression “polyalkylene-polyamine” designates a compound with at least two alkylene groups and at least two amino groups selected from primary or secondary amino groups. Preferred polyalkylene-polyamines comprise two to 10 alkylene groups and 3 to 11 amino groups selected from primary or secondary amino groups. The molecular weight of the polyalkylene-polyamines is preferably lower than 1000 g/mol more preferably lower than 700 g/mol.

In case of 1,2 propylenediamine and 1-amino propan-2-ol and hydrogen as starting materials dimethyl-diethylene-triamines selected from

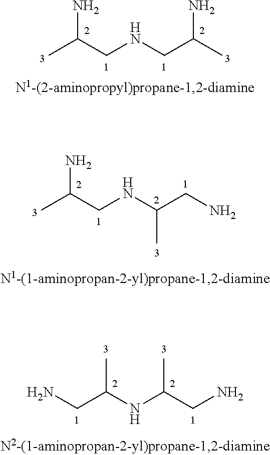

or any mixture thereof are obtained.

The compounds of N¹-(2-aminopropyl)propane-1,2-diamine, N¹-(1-aminopropan-2-yl)propane-1,2-diamine and N²-(1-aminopropan-2-yl)propane-1,2-diamine or any mixture thereof are herein collectively referred to as dimethyl-diethylene-triamines (shortly DMDETAs).

The dimethyl-diethylene-triamines obtained may be starting material for a subsequent reductive amination by reacting with unconsumed 1-amino propan-2-ol and hydrogen, thus obtaining a further isomeric mixture comprising, for example, a dimethyl-tetraethylene-pentamine of formula

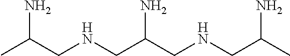

or any structural isomers thereof.

The above compound, any structural isomer thereof and any mixture of these compounds are herein collectively referred to as dimethyl-tetraethylene-pentamines.

A further reductive amination of dimethyl-tetraethylene-pentamines leads to an isomeric mixture comprising, for example, a compound of formula

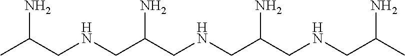 [SMILES: CC(N)CNCC(N)CNCC(N)CNCC(C)N]

or any structural isomers thereof.

The above compound, any structural isomer thereof and any mixture of these compounds are herein collectively referred to as dimethyl-hexaethylene-heptamines.

Product mixtures comprising the first reductive amination product and sequential reductive amination products of the alkylene-diamine and the hydroxyalkylene-amine may be obtained by the process of this invention.

In case of 1,2 propylenediamine and 1-amino propan-2-ol product mixtures comprising dimethyl-diethylene-triamines and the sequential reductive amination products such as dimethyl-tetraethylene-pentamines and dimethyl-hexaethylene-heptamines may be obtained.

The content of the first reductive amination product and the sequential reductive amination products depends particularly from the stoichiometric ratio of the starting materials and the reaction conditions. A shorter reaction time or lower temperature or reduced amount of the hydroxyalkylene-amine or any combination thereof lead to less sequential reductive amination products.

In a preferred embodiment, the stoichiometric ratio of the starting materials and the reaction time are adjusted to obtain a mixture of polyalkylene-polyamines that comprises at least 50% by weight, preferably at least 60% by weight, more preferably at least 70% and most preferably at least 90% by weight of dimethyl-diethylene-triamines, based on all polyalkylene-polyamines.

Furthermore, the dimethyl-diethylene-triamines obtained are preferably an isomeric mixture of

1 to 49% by weight of N1-(2-aminopropyl)propane-1,2-diamine,
50 to 98% by weight of N1-(1-aminopropan-2-yl)propane-1,2-diamine and
1 to 49% by weight of N2-(1-aminopropan-2-yl)propane-1,2-diamine
based on 100% by weight of dimethyl-diethylene-triamines.

To the process

In the process, the alkylene-diamine to the hydroxy-alkylene-amine may be used, for example, in a molar ratio of 1:0.1 to 0.1:1.

In preferred embodiment, an excess of the hydroxyalkylene-amine is avoided and the molar ratio of the alkylene-diamine to the hydroxy-alkylene-amine is 1:0.1 to 1:1, notably 1:0.1 to 1:0.8.

The reaction is preferably performed at a pressure of 10 to 500 bars, more preferably at a pressure of 50 to 300 bars, notably at a pressure of 100 to 250 bars and most preferably at a pressure of 120 to 250 bars.

As the reaction is performed in presence of hydrogen, the reactor is preferably pressurized with hydrogen or mixtures of hydrogen with inert gases like nitrogen to obtain the pressure above. In a preferred embodiment, pure hydrogen is used. Any oxygen should be removed from the reactor before hydrogen is introduced into the reactor, for example by feeding an inert gas, first.

The alkylene-diamine, hydroxyalkylen-amine and hydrogen may be reacted in presence of ammonia, as ammonia supports the reductive amination reaction. In the presence of ammonia, the selectivity of the reaction (ration of DMDETA versus piperazine by-products) is improved. Preferably, the amount of ammonia added to the reaction is in the range of 5 to 500% by weight, more preferably in the range of 10 to 100% by weight based on the hydroxyalkylene-amine.

The reaction is performed in presence of an heterogenous catalyst comprising copper. The word heterogenous catalyst designates a solid catalyst, preferably in form of particles, in contact with a liquid or gaseous medium, which is usually the reaction mixture comprising the starting materials and any products already obtained.

The heterogeneous catalyst may be a supported or an unsupported catalyst. A supported catalyst comprises copper and optionally other catalytically active metals on a support. Suitable supports are, for example, calcium carbonate, silicon dioxide, zirconium dioxide or aluminum oxide.

A suitable unsupported catalyst is, for example, Raney copper or solid, unsupported particles of copper together with other catalytically active metals.

The heterogeneous catalyst is preferably a supported catalyst.

The heterogeneous catalyst may comprise other catalytically active metals which are preferably selected from nickel, cobalt, palladium, platinum, rhodium, iridium, manganese, tin or ruthenium or chromium.

The heterogeneous catalyst may comprise copper and other catalytically active metals in elementary form or in form of chemical compounds. Chemical compounds may comprise the catalytically active metal in ionic form (salts) or covalently bonded form, as is often the case in metal oxides. In the following, the term metal encompasses elemental metals and also metals present in chemical compounds either in ionic form or in covalently bonded form.

Preferably, the content of copper in the heterogeneous catalyst is at least 10% by weight, more preferably at least 30% by weight, more preferably at least 40% by weight, more preferably at least 50% by weight, notably at least 60% by weight and most preferably at least 80% by weight based on the total weight of all catalytically active metals of the catalyst, whereby in case of chemical compounds such as salts or oxides, the metal fraction of such compounds is considered, only. Any metals that form part of the support, such as alumina in alumina oxide or calcium in calcium carbonate are not considered as catalytically active metals.

When oxides or other chemical compounds of the active metals are used, a reduction of these compounds to the elementary metals may be accomplished, notably at higher temperatures and often in the presence of hydrogen. This may occur simultaneously with beginning of the reaction or it may be carried out beforehand in a separate step.

The catalyst may be dispersed in the reaction mixture or may be installed in the reactor for example as fixed bed.

The reaction may be performed as batch process, semi-continuously or continuously. In a batch process all starting materials are added to the reactor. In a semi-continuous process at least one of the starting materials is added completely to the reactor and at least one is fed in course of the reaction. In a continuous process all starting materials are continuously fed and products are continuously withdrawn from the reactor.

In a batch or semi batch process, the heterogeneous catalyst is preferably used in an amount of 0.1 to 10 parts by weight per 100 parts by weight of the alkylene-diamine and hydroxyalkylene-amine. In a continuous process, the total amount of alkylene-diamine and hydroxyalkylene-amine which is continuously fed to the reactor per hour is preferably 0.05 to 5 kilogram per 1 kg of catalyst located in the reactor.

The reaction may be monitored via gas chromatography. The yield of a product obtained corresponds to the area of the corresponding peak compared to the area of all peaks.

By-products which may be obtained by reacting 1,2 propylene-diamine and 1-amino propan-2-ol are piperazine derivatives. For example, N¹-(1-aminopropan-2-yl)propane-1,2-diamine may undergo an intramolecular condensation reaction to give a piperazine derivative

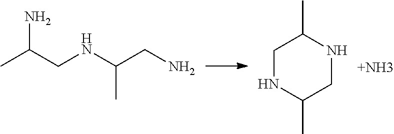

The obtained product mixture may be purified by usual methods, notably by distillation at reduced pressure. Unconsumed starting materials, by-products like piperazine derivatives are removed and higher molecular weight products such as sequential reductive amination products remain as residue.

The process of this invention is an easy and very economic process to produce polyalkylene-polyamines. The process is very selective. It is an advantage of the process that the formation of piperazine derivatives can be suppressed and satisfying yields of the polyalkylene-polyamines are obtained. Sequent reductive amination products are formed in low amounts, only, if any. The selectivity with respect to dimethyl-diethylene-triamines is high.

### EXAMPLES

### Example 1 to 12

An autoclave was charged with 20 g of a supported catalyst in form of a fixed-bed catalyst or with 10 g of Raney catalyst (washed with THF) under an atmosphere of nitrogen. 32 g 1-aminopropan-2-ol (˜90:10 mixture with 2-aminopropan-1-ol), shortly MIPOA, and 60 g propane-1,2-diamine, shortly 1,2-PDA were added, the autoclave was sealed and pressurized with hydrogen to 10 bar. In Examples 8 to 10 also 5 g ammonia were added to the reaction mixture. The reaction mixture was stirred and heated to the temperature (T) listed in the Table below. After the temperature was reached, the pressure was adjusted with hydrogen to the pressure (P) listed in the Table below and the mixture was stirred for 12 h. A sample was taken after 6 h and analyzed by GC. The process has been performed with different catalysts. Results (GC area-%) are listed in the Table below.

Examples 3,4,11 and 12 are comparison examples. The catalyst material used for Examples 1 and 5 contained 10% by weight of cobalt, calculated as CoO, 10% by weight of nickel, calculated as NiO, and 4% by weight of copper, calculated as CuO, remainder Al₂O₃, which corresponds to a content of copper of 18% by weight based on the total weight of all catalytically active metals of the catalyst (only considering the metal fraction of the compounds of the active mass). The catalyst material used for Examples 2 and 6 to 8 contained 51% by weight of copper, calculated as CuO, remainder Al₂O₃, (since the active mass of this catalyst comprises no further metals in addition to copper, the content of copper based on the total weight of all catalytically active metals of the catalyst is 100%). The catalyst material used for Examples 9 and 10 contained 45% by weight of copper, calculated as CuO and 46% by weight of chromium, calculated as Cr₂O₃, remainder BaO promoter, which corresponds to a content of copper of 53% by weight based on the total weight of all catalytically active metals of the catalyst (only considering the metal fraction of the compounds of the active mass).

TABLE
catalysts and results of examples 1 to 12

| | | | | sum of | | | |
|---|---|---|---|---|---|---|---|
| | | | | residual | | | |
| | | | | 1,2 PDA + | sum of | Sum of | |
| | | | | MIPOA | piperazine | DMDETA | Ratio |
| example | catalyst | T (° C.) | P (bar) | (%) | (%) | (%) | DMDETA:piperazine |
| 1 | Co—Ni—Cu | 200 | 200 | 23 | 65 | 3 | 0.05 |
| | on Al₂O₃ | | | | | | |
| 2 | Cu | 200 | 200 | 44 | 11 | 31 | 2.8 |
| | on Al₂O₃ | | | | | | |
| 3 | Raney Ni | 200 | 200 | 8 | 74 | 0 | 0 |
| 4 | Raney Co | 200 | 200 | 1 | 73 | 0.2 | 0.0003 |
| 5 | Co—Ni—Cu | 180 | 200 | 46 | 33 | 13 | 0.4 |
| | on Al₂O₃ | | | | | | |
| 6 | Cu | 180 | 200 | 75 | 2 | 19 | 9.5 |
| | on Al₂O₃ | | | | | | |
| 7 | Cu | 180 | 120 | 77 | 3 | 17 | 5.7 |
| | on Al₂O₃ | | | | | | |
| 8 | Cu | 180 | 120 | 73 | 3 | 20 | 6.7 |
| | on Al₂O₃ | | | | | | |
| 9 | Cu/Cr | 180 | 120 | 77 | 3 | 16 | 5.3 |
| 10 | Cu/Cr | 200 | 200 | 65 | 8 | 20 | 2.5 |
| 11 | Raney Ni | 180 | 200 | 32 | 43 | 5 | 0.12 |
| 12 | Raney Co | 180 | 200 | 31 | 57 | 1 | 0.02 |

Raney Co was obtained from Grace (Grace 2724), Raney Ni was obtained from BASF (H1-50), the catalysts on Al₂O₃ support were BASF products.

### Example 13

### Continuous Process for the Preparation of DMDETA

A tubular reactor was filled with 600 mL of a Cu catalyst (as used for Example 6). For catalyst activation, the catalyst was heated to a temperature of 180° C. to 200° C. under a stream of nitrogen at atmospheric pressure. Hydrogen was carefully dosed into the nitrogen stream at atmospheric pressure to control the exotherm of the activation. Eventually, pure hydrogen was passed over the catalyst at atmospheric pressure and at a temperature of 200° C. for 6 h. After catalyst activation, the reactor was pressurized to 200 bar with H₂ and fed continuously with 100 g/h 1-aminopropan-2-ol (˜90:10 mixture with 2-aminopropan-1-ol), 200 g/h propane-1,2-diamine, 80 g/h NH₃ and 100 NL/h H₂ at a temperature of 180 to 220° C. The product stream was depressurized to atmospheric pressure and collected. The collected crude product was purified by distillation under reduced pressure to obtain an ˜6:87:6 isomeric mixture of N¹-(2-aminopropyl)propane-1,2-diamine, N¹-(1-aminopropan-2-yl)propane-1,2-diamine and N²-(1-aminopropan-2-yl)propane-1,2-diamine in >99% purity (GC-area-%, sum of isomers).

1: A process to produce polyalkylene-polyamines, the process comprising:
reacting an alkylene-diamine, a hydroxyalkylene-amine, and hydrogen in presence of a heterogenous catalyst comprising copper, to obtain the polyalkylene-polyamines.

2: The process according to claim 1, wherein the alkylene-diamine is a compound of formula I
H₂N—X—NH₂,
with X representing a linear or branched alkylene group with 2 to 10 carbon atoms.

3: The process according to claim 2, wherein the alkylene-diamine of formula I is 1,2 propylenediamine of the following formula

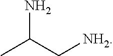

4: The process according to claim 1, wherein the hydroxyalkylene-amine is a compound of formula II
HO—Y—NH₂,
with Y representing a linear or branched alkylene group with 2 to 10 carbon atoms.

5: The process according to claim 4, wherein the hydroxyalkylene-amine of formula II is 1-amino propan-2-ol of the following formula

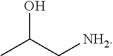

6: The process according to claim 1, wherein the polyalkylene-polyamines obtained comprise dimethyl-diethylene-triamines selected from the group consisting of 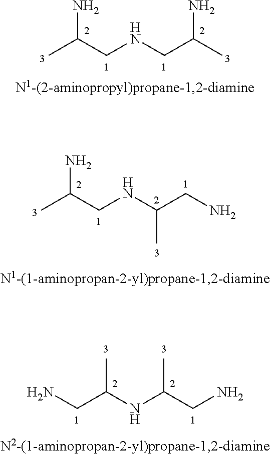
and any mixture thereof.

7: The process according to claim 1, wherein the catalyst comprises at least 30% by weight of copper, based on all catalytically active metals in the catalyst, calculated as elementary metals.

8: The process according to claim 1, wherein the catalyst is a supported catalyst and a support is selected from the group consisting of calcium carbonate, silicon dioxide, zirconium dioxide, and aluminum oxide.

9: The process according to claim 1, wherein the alkylene-diamine, the hydroxyalkylene-amine, and hydrogen are reacted in presence of ammonia.

10: The process according to claim 1, wherein the process is performed continuously.
